(19) United States
(12) Patent Application Publication
(10) Pub. No.: US 2023/0301634 A1
(43) Pub. Date: Sep. 28, 2023

(54) NON-INVASIVE AND NON-OBTRUSIVE MEAN ARTERIAL PRESSURE ESTIMATION

(71) Applicant: Analog Devices, Inc., Wilmington, MA (US)

(72) Inventor: Joohyun SEO, Medford, MA (US)

(21) Appl. No.: 18/189,707

(22) Filed: Mar. 24, 2023

Related U.S. Application Data
(60) Provisional application No. 63/324,050, filed on Mar. 26, 2022.

(51) Int. Cl. A61B 8/08; A61B 8/04; A61B 8/06; A61B 8/00

(52) U.S. Cl. CPC A61B 8/5223; A61B 8/04; A61B 8/06; A61B 8/461

(57) ABSTRACT

Aspects described herein relate to estimating mean arterial pressure (MAP) of a living being. The estimating may include obtaining pulsatile arterial blood pressure (pABP) waveform, obtaining arterial blood flow (ABF) waveform, identifying a set of segments of the pABP waveform in steady state, identifying a set of segments of the ABF waveform in steady state, and estimating the MAP based on the identified segment of the pABP waveform in steady state and the identified segment of the ABF waveform in steady state

### CLAIM OF PRIORITY UNDER 35 U.S.C. § 119

The present Application for Patent claims priority to Provisional Patent Application No. 63/324,050, entitled “NON-INVASIVE ULTRASOUND-BASED MEAN ARTERIAL PRESSURE ESTIMATION” filed Mar. 26, 2022, which are assigned to the assignee hereof and hereby expressly incorporated by reference herein for all purposes.

### TECHNICAL FIELD OF THE DISCLOSURE

The present invention relates to the field of biomedical devices, in particular to non-invasive and non-obtrusive mean arterial pressure estimation.

### BACKGROUND

Cardiovascular diseases are disorders of the heart and blood vessels. Examples of cardiovascular diseases include coronary heart disease, cerebrovascular disease, peripheral arterial disease, rheumatic heart disease, congenital heart disease, deep vein thrombosis, and pulmonary embolism. One tool for monitoring cardiovascular systems is the electrocardiogram (ECG), which records body surface potential and assesses electrical functionality of the heart non-invasively. Other non-invasive tools include: ultrasonography, computed tomography, angiography, magnetic resonance imaging, and Doppler spectrogram.

One key physiological measurement of the cardiovascular system is blood pressure, or arterial blood pressure (ABP). Often, systolic and diastolic blood pressure are sampled by a sphygmomanometer, e.g., an inflatable cuff around the arm with auscultation by a stethoscope. However, the under-sampled measurements are insufficient to truly represent the dynamic behavior of the cardiovascular system.

A complete ABP waveform can be generated using an invasive tool, where a pressure sensor reads the ABP waveform in the radial or femoral artery is accessed through arterial catheterization. This tool is only available in intensive care units, and is not practical for clinical or at home uses due to its invasive nature.

A complete ABP waveform can be a powerful predictor for cardiovascular diseases. Additionally, the waveform can provide useful information on the cardiovascular system. Some efforts have been made to measure the ABP waveform non-invasively, but their reliability and practicality remain limited.

### SUMMARY

The following presents a simplified summary of one or more aspects in order to provide a basic understanding of such aspects. This summary is not an extensive overview of all contemplated aspects, and is intended to neither identify key or critical elements of all aspects nor delineate the scope of any or all aspects. Its sole purpose is to present some concepts of one or more aspects in a simplified form as a prelude to the more detailed description that is presented later.

In an aspect, a method to estimate mean arterial pressure (MAP) of a living being is provided that includes obtaining pulsatile arterial blood pressure (pABP) waveform, obtaining arterial blood flow (ABF) waveform, identifying a set of segments of the pABP waveform and the ABF waveform in time that are in steady state, estimating the MAP based on the identified set of segments in time that are in steady state in both the pABP waveform and the ABF waveform, and displaying an indication of the estimated MAP.

In another aspect, a biomedical device is provided that includes a storage, and a digital processor coupled to the storage. The digital processor is configured to obtain pulsatile arterial blood pressure (pABP) waveform, obtain arterial blood flow (ABF) waveform, identify a set of segments of the pABP waveform and the ABF waveform in time that are in steady state, and estimate the MAP based on the identified set of segments in time that are in steady state in both of the pABP waveform and the ABF waveform.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

To provide a more complete understanding of the present disclosure and features and advantages thereof, reference is made to the following description, taken in conjunction with the accompanying figures, wherein like reference numerals represent like parts, in which:

FIG. 1 is a flow diagram illustrating an example of a method to estimate mean arterial blood pressure, according to some embodiments of the disclosure;

FIG. 2 illustrates examples of signals being processed to identify steady state, according to some embodiments of the disclosure;

FIG. 3 is a flow diagram illustrating an example of another method to estimate mean arterial blood pressure, according to some embodiments of the disclosure;

FIG. 4 illustrates examples of signals being processed to determine peripheral resistance, according to some embodiments of the disclosure; and

FIG. 5 illustrates an example of an ultrasound system that can generate an ABP waveform without calibration, according to some embodiments of the disclosure.

### DESCRIPTION OF EXAMPLE EMBODIMENTS OF THE DISCLOSURE

Non-Invasive Measurements and Need for Calibration

Various modalities can be used to measure arterial blood flow, such as ultrasound, or ultrasonography, magnetic resonance imaging (MRI), or other non-invasive and/or non-obtrusive modalities. Non-obtrusive can refer to there being no need of applying significant pressure to a living being for the modality. Obtrusive modalities may include, for example, using a cuff or like tonometry or force-coupled ultrasound elastography techniques, etc. Compared to other modalities, ultrasound is widely available, less expensive, portable, and does not have ionizing radiation. MRI is also widely available and can be somewhat portable. Ultrasound, MRI, or other modalities can produce or measure arterial flow (e.g., velocity or flow rate) waveform (referred herein as the arterial blood flow (ABF) waveform), and arterial distension (diameter) waveform. Pulse wave velocity (PWV) can also be estimated, which can provide a representation of the characteristic impedance of the conduit artery at the measurement site (e.g., when considered in view of blood density and cross-sectional area of the artery). A pulsatile ABP waveform (referred herein as the pABP waveform) can be estimated from the velocity and diameter waveforms (or a derivation thereof).

Some techniques have been able to extract the absolute ABP waveform with calibration. These techniques measure local PWV to get arterial compliance, and estimate ABP waveform from arterial distension waveform to obtain pABP and eventually to absolute ABP waveform through calibration.

In some techniques, arterial compliance (dA/dP) is independently estimated, for example through PWV. Incremental distension (dA) and PWV can be combined to derive pulsatile pressure versus time (dP). One exemplary equation (rewritten from the Bramwell-Hill equation that relates pressure (P) and cross-sectional area (A)) to derive pulsatile pressure over time is as follows:

$$dp = ρ P W {V}^{2} \frac{dA}{A} \qquad ( 1 )$$

ρ is the density of blood. Reformulating the above equation can yield:

$$P ( t ) - MAP = ρ P W {V}^{2} ln \frac{A ( t )}{{A}_{m e a n}} \qquad ( 2 )$$

The measurements, i.e., distention and PWV, can yield the pABP waveform corresponding to

$$ρ {PWV}^{2} ln \frac{A ( t )}{{A}_{mean}} .$$

P(t) corresponds to the absolute ABP waveform. The constant or offset (referred to herein as DC offset), i.e., MAP is unknown. To obtain P(t), the absolute ABP waveform, MAP can be determined through a calibration process, such that the waveform

$$ρ P W {V}^{2} ln \frac{A ( t )}{{A}_{mean}}$$

can be adjusted based on the DC offset MAP.

Therefore, calibration may be used in this technique because the DC offset level (i.e., MAP) can be measured by other mechanisms to obtain the absolute ABP waveform. Specifically, the DC offset can be obtained so that the waveform, i.e.,

$$ρ {PWV}^{2} ln \frac{A ( t )}{{A}_{mean}} ,$$

can be shifted with the appropriate DC offset to yield the absolute pressure over time, i.e., P(t). The offset of the absolute ABP waveform, i.e., MAP, corresponds to mean arterial pressure (MAP), and may be separately obtained in this technique using the diastolic blood pressure (DPB) from a sphygmomanometer.

One technical problem that may be solved in accordance with aspects described herein can be to determine MAP (i.e., the DC offset in Equation (2)) without necessarily requiring a separate measurement. This can enable the absolute ABP waveform to be generated without calibration.

Steady State Ohm's Law of Arterial Hemodynamics

From a systems analysis standpoint, the cardiovascular system can be analyzed in its “steady state” where no energy storing elements change its state after completion of a cycle. When examining the system in “steady state”, certain relationships of different physiological parameters can hold true.

In “steady state” at a full body level, the MAP of the whole body is equal to total peripheral resistance (TPR) times cardiac output (CO). Cardiac output can be defined as the time-average value of volumetric flow measurement of blood coming out from the heart. Phrased mathematically:

MAP_{whole body}=TPR×CO (3)

In “steady state” fora particular arterial branch (e.g., major conduit artery such as the carotid, the iliac, the femoral, and the brachial), the MAP of the arterial branch can be equal (or substantially equal) to the peripheral resistance (PR) downstream of the arterial branch times the time-average volumetric flow during the steady state. Phrased mathematically:

MAP_{arterial branch}=PR×time-average volumetric flow (4)

Note that MAP_{full body} and MAP_{arterial branch} are expected to differ by hydrostatic pressure difference. Time-average volumetric flow can be defined as the time-average value of volumetric flow measurement of blood through the arterial branch.

In “steady state”, PR is equal to change in MAP, or ΔMAP divided by change in mean arterial flow (MAF), or ΔMAF. MAF can be equivalent to time-averaged volumetric flow. Phrased mathematically:

PR=ΔMAP/ΔMAF (5)

The ABP waveform can show slow variation due to respiration, baroreceptor reflex (Mayer wave). With ultrasound measurements and estimation, it is possible to measure ΔMAP and ΔMAF at the steady state.

Identifying steady state is not trivial. Assuming all pulse wave behavior is settled down at the end-diastole, the beat-to-beat difference in diastolic blood pressure, if small enough, can indicate that the system is in a steady state. The reasoning is that if after one cycle, the diastolic blood pressure (DBP) returned to the same (initial) state, then the system is in a steady state during that cycle. The state variable is assumed to be pressure and the only (or at least dominant) energy storage mechanism is compliance of the elastic artery. The beat-to-beat difference, or ΔDBP, phrased mathematically, can be:

ΔDBPᵢⱼ=DBPⱼ−DBPᵢ (6)

DBPᵢ is the diastolic blood pressure at a given beat i. The difference ΔDBPᵢⱼ can be an absolute value of the difference in two diastolic blood pressures: DBP at beat j and DBP at beat i.

Estimating MAP at Steady State

Based on this insight, it is possible to analyze the waveforms obtained or obtained from ultrasound to extract data associated with steady state. Based on the extracted data associated with steady states, mean pulsatile arterial pressure (MpAP) and MAF at steady states can be determined. When a line is fitted to MpAP and MAF pairs, the slope of the fitted line yields the quantity, ΔMpAP/ΔMAF. In this instance, ΔMpAP is equivalent to ΔMAP since they both correspond to differential values of the same waveform. Therefore, ΔMpAP/ΔMAF also yields ΔMAP/ΔMAF, the quantity seen in Equation (5). In other words, from the slope, i.e., ΔMpAP/ΔMAF, PR can be estimated based on Equation (5). From PR and time-averaged volumetric flow, MAP_{arterial branch} (referred herein also as MAP) can then be estimated based on Equation (4).

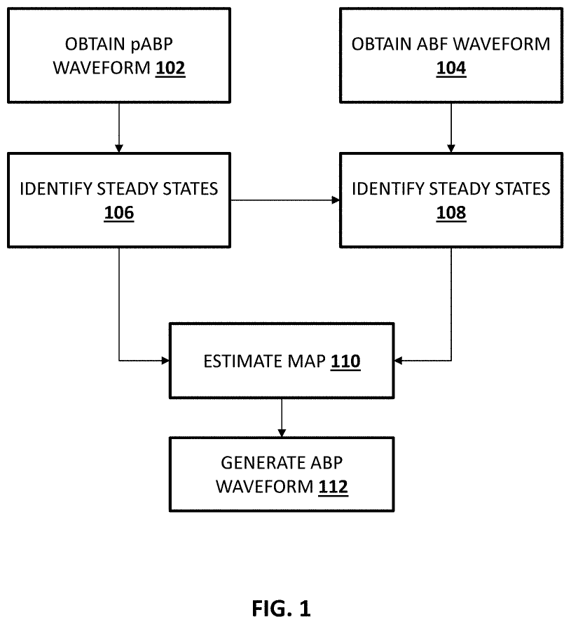

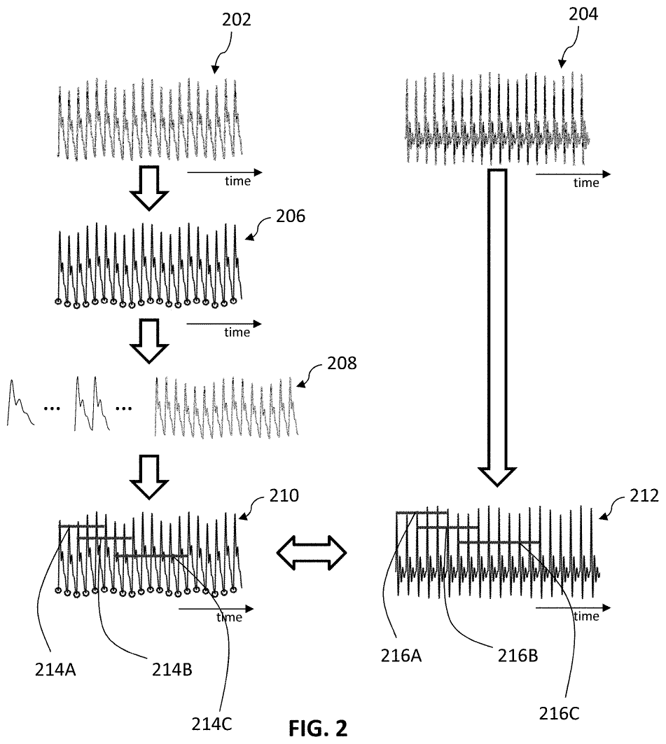

FIG. 1 is a flow diagram illustrating an example of a method to estimate mean arterial blood pressure (i.e., MAP_{arterial branch}), according to some embodiments of the disclosure. In an example, the method of FIG. 1 can be performed by a processor, such as digital processor 518, which may include processing corresponding instructions stored in memory, such as storage 516 described herein. FIG. 2 illustrates examples of signals being processed to identify steady state, according to some embodiments of the disclosure. The parts of the method in FIG. 1 are described with reference to FIG. 2.

In 102, pulsatile arterial blood pressure (pABP) waveform is obtained. An exemplary pABP waveform is shown as 202 in FIG. 2. In some embodiments, an ultrasound system, MRI, or other system can non-invasively and/or non-obtrusively measure distension information of an artery (e.g., arterial distension (diameter) waveform) and can generate PWV information and blood flow (e.g., from measured arterial flow (velocity) waveform) in the artery. The distension information and the PWV information can be processed to generate the pABP waveform, e.g.,

$$ρ PW {V}^{2} ln \frac{A ( t )}{{A}_{m e a n}}$$

in Equation (2). The pABP waveform may not completely represent the (absolute) ABP waveform, e.g., P(t) in Equation (2), because the DC offset, corresponding to MAP in Equation (2), may be absent or unknown at this time.

In 104, ABF waveform is obtained. An exemplary ABF waveform is shown as 204 in FIG. 2. The ABF waveform is the arterial flow (velocity or flow rate) waveform, which can be non-invasively and/or non-obtrusively measured by the ultrasound system, MRI, or other systems.

In 106, steady states in the pABP waveform can be identified based on identifying multiple sets of segments of cardiac cycles of the pABP waveform in steady state, with various levels of MAF and MpAP. Cardiac cycles, as used herein, are defined as periods between each end-diastole. In some embodiments, the pABP waveform can be processed to identify end-diastole points in the waveform, which correspond to local minimums in the pABP waveform. Examples of end-diastole points are shown as circled points in waveform 206 of FIG. 2. The end-diastole points can be points used to segment the pABP waveform into a plurality of segments corresponding to beats, and the end-diastole points can represent the DBP of the beats. Steady state is defined to be a scenario where beat-to-beat difference in DBP is sufficiently small or within a threshold, which can indicate that the system came back to more or less the same or substantially similar (initial) state after one cycle.

The difference between pressure data points at end-diastole can be defined by Equation (6). In 106, sets of segments can be identified to meet a steady state criteria, where pairwise differences in pressure at end-diastole are all less than a threshold. The sets of segments can be identified by considering all segments at the units of cardiac cycles starting and ending with end-diastole, as shown by waveforms 208 of FIG. 2. More specifically, in an example, sets of segments can correspond to sets of contiguous segments can be identified where all combinations of pairwise differences between two pressure data points at end-diastole corresponding to two segments in the set of contiguous segments in the pABP waveform are less than a threshold. An algorithm can iterate through all possible pairwise combinations between beat i and beat j to determine the differences, DBPⱼ−DBPᵢ, and assess if all of the differences are all less than the threshold. In one example, if all of the differences of the possible pairwise combinations between beat i and beat j are all less than the threshold, then contiguous segments from beat i to beat j−1 are declared as steady state. In one specific non-limiting example, the threshold can be 0.5 millimeters of mercury (mmHg), but other values are envisioned by the disclosure. Conversely, for example, if not all pairwise combinations of beat-to-beat differences between beat i and beat j are less than the threshold, then the segments between beat i and beat j are considered not to be in steady state. The algorithm can identify one or more sets of segments that meet this criteria, and therefore the algorithm can identify one or more steady states.

In an example, a given set of segments belonging to a steady state can include two or more contiguous segments. For example, depending on the noise level of the pABP waveform and ABF waveform, the algorithm may impose that the number of contiguous segments has to exceed or equal to a minimum number in order for the set of segments to be considered to belong to steady state. It is possible that some of the sets of contiguous segments overlap each other in the pABP waveform in time, or share segments with each other. Examples of sets of contiguous segments belonging to three steady states are illustrated as 214A, 214B, and 214C of pABP waveform 210 in FIG. 2.

Other algorithms to identify steady states are envisioned by the disclosure, so long as they can identify data that belong to steady states, where the system has returned to more or less the same state.

In 108, steady states in the ABF waveform can be identified based on identifying multiple sets of contiguous segments of cardiac cycles of ABF waveform in steady state. Specifically, the steady states can be identified based on identifying the sets of contiguous segments of cardiac cycles of ABF waveform that correspond to the sets of identified contiguous segments of the cardiac cycles of pABP waveform. The ABF waveform can be segmented into beats or segments separated by end-diastole points. The segments of the pABP waveform may have a 1:1 correspondence with the segments in the ABF waveform. In this example, the contiguous segments identified in the pABP waveform can be used to identify corresponding contiguous segments of the ABF waveform in steady state. Examples of sets of contiguous segments belonging to three steady states are illustrated as 216A, 216B, and 216C of ABF waveform 212 in FIG. 2.

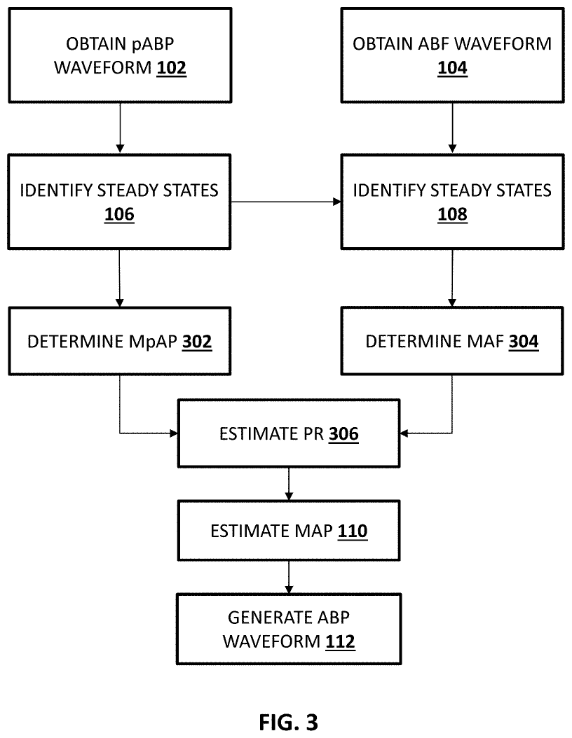

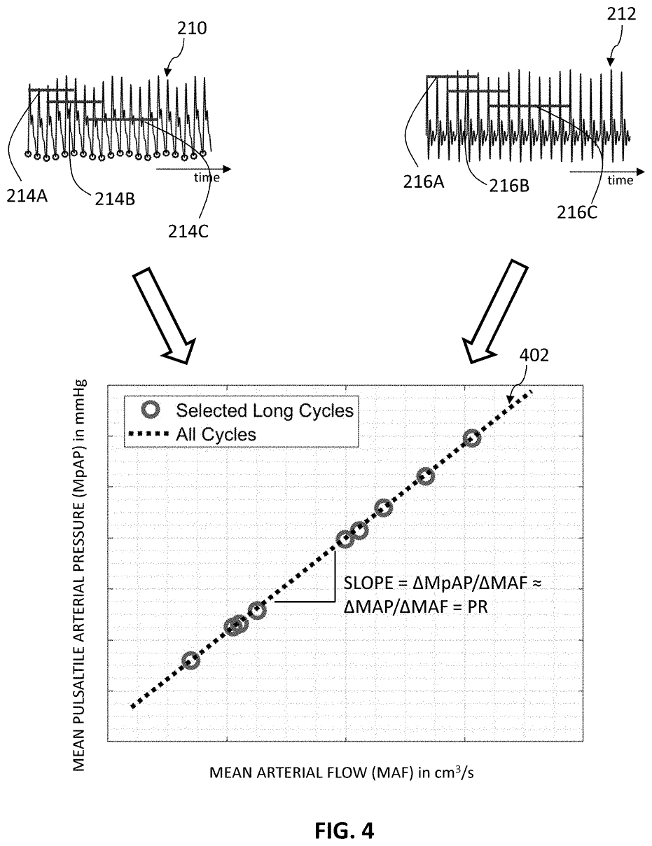

In 110, the MAP can be estimated based on the identified contiguous segments of the cardiac cycles of pABP waveform in steady state and the identified contiguous segments of the cardiac cycles of the ABF waveform in steady state. The steady state condition can allow for relationships in Equations (4) and (5) to hold true. Data points from the steady states of the pABP waveform and the ABF waveform can be used to estimate the MAP. An example of a method is described in further detail with FIGS. 3 and 4.

In 112, the absolute ABP waveform, e.g., P(t) in Equation (2), can be generated using the MAP. For instance, the pABP waveform can be level shifted by the MAP, which corresponds to the DC offset in Equation (2)).

Estimating the PR Using the Steady State Relationships

FIG. 3 is a flow diagram illustrating another example of a method to estimate MAP, according to some embodiments of the disclosure. In an example, the method of FIG. 3 can be performed by a processor, such as digital processor 518, which may include processing corresponding instructions stored in memory, such as storage 516 described herein. FIG. 4 illustrates examples of signals being processed to determine PR, according to some embodiments of the disclosure. The parts of the method in FIG. 3 are described with reference to FIG. 4.

Upon identifying steady states in 106, MpAP can be calculated for each identified contiguous segments of the cardiac cycles of the pABP waveform in 302. The MpAP can be the average value of the pressure data points in a given set of contiguous segments of the pABP waveform in steady state. Accordingly, for each steady state, a MpAP value can be calculated.

Upon identifying steady states in 108, MAF can be calculated for each identified contiguous segment of the cardiac cycles of the ABF waveform in 304. The MAF can be the average value of the flow data points in a given set of contiguous segments of the ABF waveform in steady state. Accordingly, for each steady state, a MAF value can be calculated.

In 306, the PR can be estimated from the pairs of MpAP value and MAF value corresponding to each steady states. In some embodiments, a slope of a best fit line relating the MAF versus the MpAP can be determined, and the slope can represent the PR. From the pairs of MpAP value and MAF value, a least squares line fitting can be performed, and the slope of the line can yield ΔMpAP/ΔMAF, which corresponds to the PR. As seen in the example in FIG. 4, pairs of the pairs of MpAP value and MAF value of “selected long cycles” (e.g., the sets of contiguous segments in steady state) can be plotted (shown as circles along line 402 in FIG. 4), and a line 402 can be fitted to the pairs. The slope of line 402 can yield ΔMpAP/ΔMAF, which can be equivalent to ΔMAP/ΔMAF=PR.

In 110, the MAP can be estimated, e.g., using on Equation (4), based on the PR in 306 and a time-averaged volumetric flow determined from the ABF waveform in 104 and illustrated in waveform 204 of FIG. 2. In some cases, the time-averaged volumetric flow can be the mean value of (e.g., the entirety of) the ABF waveform 204. In some cases, the time-averaged volumetric flow can be based on the mean value of a set of contiguous segments in the ABF waveform 204 identified to be at steady state (e.g., 216A, 216B, or 216C). Specifically, the MAP can be estimated by multiplying the PR and the time-averaged volumetric flow. In some embodiments, the time-averaged volumetric flow can be calculated based on an average of the data points of the ABF waveform.

Ultrasound-Based System to Generate an ABP Waveform without Calibration

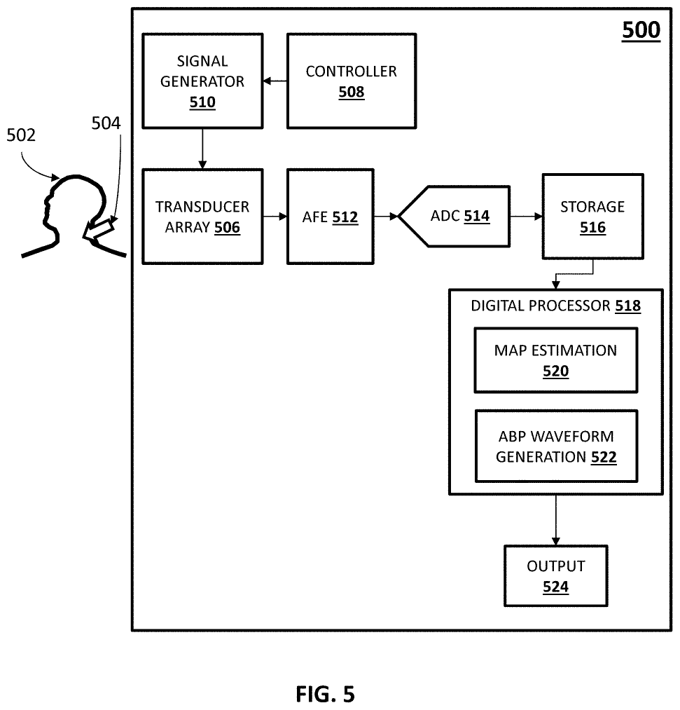

FIG. 5 illustrates an exemplary ultrasound system 500 or other biomedical device that can generate an ABP waveform without calibration, according to some embodiments of the disclosure. The ultrasound system 500 can be used on a living being 502 to make measurement of an artery (e.g., carotid as indicated by arrow 504). The ultrasound system 500 can include a transducer array 506, which is driven by a signal generator 510. A controller 508 can be provided to control the signal generator, e.g., to enable beamforming. An analog front end (AFE) 512 can be provided to receive and process analog signals from the transducer array 506. The analog signals from the AFE 512 can be digitized by analog-to-digital converter 514 to generate digital signals. Once in the digital domain, the digital signals can be stored in storage 516 (e.g., one or more non-transitory computer readable media). The storage 516 can also store instructions for executing the processing described herein. The processing described herein to estimate MAP and generate an ABP waveform can be carried out by one or more digital processors 518. The processing can be encoded in instructions stored on in storage 516, and the one or more digital processors 518 can carry out the processing when the instructions are executed by the one or more digital processors 518. For instance, the one or more digital processors 518 can implement map estimation 520 and ABP waveform generation 522, e.g., in accordance with the processes illustrated in FIGS. 1-4. Finally, the ultrasound system 500 can output the ABP waveform to an end user, e.g., via a display device of the output 524. In some cases, the ultrasound system 500 can transmit via the output 524 over a communication channel (wired or wireless) such that a system that is remote to the ultrasound system 500 can receive, process, and output the ABP waveform, a derivation of the waveform, or other related values to an end user. Other systems can carry out the processes to estimate MAP and generate an ABP waveform, as described herein, such as MRI, and can have at least some similar components as those shown in ultrasound system 500. For example, other systems can at least include the digital processor 518 to estimate MAP and generate an ABP waveform of incoming signals and/or output 524 to output the ABP waveform, derivation, or related values.

### VARIATIONS AND IMPLEMENTATIONS

Moreover, certain embodiments discussed above can be provisioned in technologies for medical imaging, patient monitoring, medical instrumentation, and home healthcare.

In the discussions of the embodiments above, various electrical components can readily be replaced, substituted, or otherwise modified in order to accommodate particular circuitry needs. Moreover, it should be noted that the use of complementary electronic devices, hardware, software, etc. offer an equally viable option for implementing the teachings of the present disclosure.

Parts of various circuitry for carrying out the methods described herein can include electronic circuitry to perform the functions described herein. In some cases, one or more parts of the circuitry can be provided by a processor specially configured for carrying out the functions described herein. For instance, the processor may include one or more application specific components, or may include programmable logic gates which are configured to carry out the functions describe herein. The circuitry can operate in analog domain, digital domain, or in a mixed signal domain. In some instances, the processor may be configured to carrying out the functions described herein by executing one or more instructions stored on a non-transitory computer medium. In some embodiments, an apparatus can include means for performing or implementing one or more of the functionalities describe herein.

The specifications, dimensions, and relationships outlined herein (e.g., the number of processors, logic operations, etc.) are offered for purposes of example and teaching. Such information may be varied considerably without departing from the spirit of the present disclosure. The specifications apply only to one non-limiting example and, accordingly, they should be construed as such. In the foregoing description, example embodiments have been described with reference to particular processor and/or component arrangements. Various modifications and changes may be made to such embodiments without departing from the scope of the disclosure. The description and drawings are, accordingly, to be regarded in an illustrative rather than in a restrictive sense.

Note that with the numerous examples provided herein, interaction may be described in terms of two, three, four, or more electrical components. However, this has been done for purposes of clarity and example only. It should be appreciated that the system can be consolidated in any suitable manner. Along similar design alternatives, any of the illustrated components, modules, and elements of the FIGURES may be combined in various possible configurations, all of which are clearly within the broad scope of this Specification. In certain cases, it may be easier to describe one or more of the functionalities of a given set of flows by only referencing a limited number of electrical elements. It should be appreciated that the electrical circuits of the FIGURES and its teachings are readily scalable and can accommodate a large number of components, as well as more complicated/sophisticated arrangements and configurations. Accordingly, the examples provided should not limit the scope or inhibit the broad teachings of the electrical circuits as potentially applied to a myriad of other architectures.

Note that in this Specification, references to various features (e.g., elements, structures, modules, components, steps, operations, characteristics, etc.) included in “one embodiment”, “example embodiment”, “an embodiment”, “another embodiment”, “some embodiments”, “various embodiments”, “other embodiments”, “alternative embodiment”, and the like are intended to mean that any such features are included in one or more embodiments of the present disclosure, but may or may not necessarily be combined in the same embodiments.

The functions related to deriving unknown impedances, illustrate only some of the possible functions that may be executed by, or within, systems illustrated in the FIGURES. Some of these operations may be deleted or removed where appropriate, or these operations may be modified or changed considerably without departing from the scope of the present disclosure. In addition, the timing of these operations may be altered considerably. The preceding operational flows have been offered for purposes of example and discussion. Substantial flexibility is provided by embodiments described herein in that any suitable arrangements, chronologies, configurations, and timing mechanisms may be provided without departing from the teachings of the present disclosure.

The following examples are illustrative only and aspects thereof may be combined with aspects of other embodiments or teaching described herein, without limitation.

Example 1 is a method to estimate mean arterial pressure (MAP) of a living being including obtaining pulsatile arterial blood pressure (pABP) waveform, obtaining arterial blood flow (ABF) waveform, identifying sets of contiguous segments of cardiac cycles of pABP waveform in steady state, identifying sets of contiguous segments of cardiac cycles of ABF waveform in steady state, and estimating the MAP based on the identified contiguous segments of the cardiac cycles of pABP waveform in steady state and the identified contiguous segments of the cardiac cycles of the ABF waveform in steady state.

In Example 2, the method of Example 1 includes where the cardiac cycles are defined as periods between each end-diastole.

In Example 3, the method of Example 1 or 2 includes where identifying the sets of contiguous segments of the cardiac cycles of the pABP waveform are in a steady state includes identifying the sets of contiguous segments where all pairwise differences between two pressure data points at end-diastole of two segments in the contiguous segments in the pABP waveform are less than a threshold.

In Example 4, the method of Example 3 includes where the threshold is 0.5 mmHg.

In Example 5, the method of any one of Examples 1˜4 includes where identifying the sets of contiguous segments of the cardiac cycles of the ABP waveform includes identifying the sets of contiguous segments of cardiac cycles of ABF waveform that corresponds to the sets of identified contiguous segments of the cardiac cycles of pABP waveform.

In Example 6, the method of any one of Examples 1-5 includes determining a mean pulsatile arterial pressure for each identified sets of contiguous segments of the cardiac cycles of the pABP waveform.

In Example 7, the method of any one of Examples 1-6 includes determining a mean arterial flow for each identified sets of contiguous segments of the cardiac cycles of the ABF waveform.

In Example 8, the method of Example 6 and 7 includes estimating peripheral resistance (PR) based on the mean pulsatile arterial pressures and the mean arterial flows.

In Example 9, the method of Example 8 includes where estimating PR based on the mean arterial pressures and the mean arterial flows includes determining a slope of a best fit line relating the mean arterial flow versus the mean arterial pressure, and the slope is the peripheral resistance.

In Example 10, the method of Example 8 or 9 includes where estimating the MAP includes estimating the MAP based on the PR and a time-averaged volumetric flow determined from the ABF waveform.

In Example 11, the method of any one of Examples 8-10 includes where estimating the MAP includes estimating the MAP by multiplying PR and a time-averaged volumetric flow determined from the ABF waveform.

In Example 12, the method of any one of Examples 1-11 includes generating an absolute arterial blood pressure (ABP) waveform by level shifting the pABP waveform by the MAP.

Example 13 is a method as illustrated by any one of FIGS. 1-4.

Example 14 is a digital processor to implement any one of the methods in Examples 1-13.

Example 15 is an ultrasound system including a transducer array, an analog front end, an analog-to-digital converter, a digital processor to implement any one of the methods in Examples 1-13, and an output to output an arterial blood pressure (ABP) waveform.

What is claimed is:

1. A method to estimate a mean arterial pressure (MAP) of a living being, comprising:
obtaining pulsatile arterial blood pressure (pABP) waveform;
obtaining arterial blood flow (ABF) waveform;
identifying a set of segments of the pABP waveform and the ABF waveform in time that are in steady state;
estimating the MAP based on the identified set of segments in time that are in steady state in both of the pABP waveform and the ABF waveform; and
displaying an indication of the estimated MAP.

2. The method of claim 1, wherein the set of segments of the pABP waveform includes cardiac cycles defined as periods between end-diastoles.

3. The method of claim 2, wherein identifying the set of segments of the pABP waveform in steady state includes:
identifying a set of contiguous segments where, for each pair of segments in the set of contiguous segments, all pairwise differences between two pressure data points at the end-diastoles of the pair of segments are less than a threshold.

4. The method of claim 3, wherein the threshold is 0.5 millimeters of mercury (mmHg).

5. The method of claim 1, wherein identifying the set of segments of the pABP waveform includes:
identifying the set of segments of the ABF waveform that corresponds to the set of segments of the pABP waveform.

6. The method of claim 1, further comprising:
determining a mean pulsatile arterial pressure for the set of segments of the pABP waveform;
determining a mean arterial flow for the set of segments of the ABF waveform; and
estimating a peripheral resistance (PR) based on the mean pulsatile arterial pressure and the mean arterial flow.

7. The method of claim 6, wherein estimating the PR based on the mean arterial pressure and the mean arterial flow includes:
determining the PR as a slope of a best fit line relating the mean arterial flow versus the mean arterial pressure.

8. The method of claim 6, wherein estimating the MAP includes:
estimating the MAP based on the PR and a time-averaged volumetric flow determined from the ABF waveform during steady state.

9. The method of claim 6, wherein estimating the MAP includes:
estimating the MAP by multiplying the PR and a time-averaged volumetric flow determined from the ABF waveform.

10. The method of claim 1, further comprising:
generating an absolute arterial blood pressure (ABP) waveform by level shifting the pABP waveform by the MAP.

11. The method of claim 1, wherein displaying the indication of the estimated MAP includes displaying the indication on a display of a biomedical device.

12. A biomedical device, comprising:
a storage; and
a digital processor coupled to the storage and configured to:
obtain pulsatile arterial blood pressure (pABP) waveform;
obtain arterial blood flow (ABF) waveform;
identify a set of segments of the pABP waveform and the ABF waveform in time that are in steady state; and
estimate a mean arterial pressure (MAP) based on the identified set of segments in time that are in steady state in both of the pABP waveform and the ABF waveform.

13. The biomedical device of claim 12, further comprising:
a display for displaying an indication of the pABP waveform.

14. The biomedical device of claim 12, wherein the segment of the pABP waveform includes cardiac cycles defined as periods between end-diastoles.

15. The biomedical device of claim 14, wherein the digital processor is configured to identify the set of segments of the pABP waveform in steady state at least in part by identifying a set of contiguous segments where, for each pair of segments in the set of contiguous segments, all pairwise differences between two pressure data points at the end-diastoles of the pair of segments are less than a threshold.

16. The biomedical device of claim 12, wherein the digital processor is configured to identify the set of segments of the pABP waveform at least in part by identifying the set of segments of the ABF waveform that corresponds to the set of segments of the pABP waveform.

17. The biomedical device of claim 12, wherein the digital processor is further configured to:
determine a mean pulsatile arterial pressure for the set of segments of the pABP waveform;
determine a mean arterial flow for the set of segments of the ABF waveform; and
estimate a peripheral resistance (PR) based on the mean pulsatile arterial pressure and the mean arterial flow.

18. The biomedical device of claim 17, wherein the digital processor is configured to estimate the PR based on the mean arterial pressure and the mean arterial flow at least in part by determining the PR as a slope of a best fit line relating the mean arterial flow versus the mean arterial pressure.

19. The biomedical device of claim 17, wherein the digital processor is configured to estimate the MAP based on the PR and a time-averaged volumetric flow determined from the ABF waveform during steady state.

20. The biomedical device of claim 12, further comprising:
a transducer array;
an analog front end for processing analog signals received from the transducer array; and
an analog-to-digital converter (ADC) for generating digital signals from the analog signals,
wherein the digital processor obtains the pABP waveform and the ABF waveform as the digital signals from the ADC converter.
